(19) United States
(12) Patent Application Publication
(10) Pub. No.: US 2026/0283621 A1
(43) Pub. Date: Sep. 24, 2026

(54) MICROROBOTIC PLATFORM FOR ENDOVASCULAR EMBOLIZATION

(71) Applicant: The Chinese University of Hong Kong, Shatin, New Territories, Hong Kong SAR (CN)

(72) Inventors: Li ZHANG, Shatin, New Territories, Hong Kong SAR (CN); Chun Ho Simon YU, Shatin, New Territories, Hong Kong SAR (CN); Dongdong JIN, Shatin, New Territories, Hong Kong SAR (CN); Kai Fung CHAN, Shatin, New Territories, Hong Kong SAR (CN)

(21) Appl. No.: 19/475,746

(22) PCT Filed: Apr. 19, 2024
(86) PCT No.: PCT/CN2024/088816

§ 371 Date: Oct. 15, 2025

Related U.S. Application Data
(60) Provisional application No. 63/461,179, filed on Apr. 21, 2023.
(60) Provisional application No. 63/533,103, filed on Aug. 16, 2023.

(51) Int. Cl. A61B 17/12; A61B 17/00; A61B 34/00; A61B 34/20; A61B 34/30; A61B 90/00; A61L 24/02; A61L 24/04

(52) U.S. Cl. CPC A61B 17/12186; A61B 17/12113; A61B 34/20; A61B 34/73; A61L 24/02; A61L 24/04; A61B 2017/00876; A61B 2034/303; A61B 2090/376; A61B 2090/378; A61B 2090/3995

(57) ABSTRACT

Provided are materials, methods, and systems particularly useful for the embolization of, e.g., a target vascular sac using microgels applied as microrobots. The microrobots include self-healing material-based microgels for on-demand controllable aggregation, a magnetic agent such as magnetite nanoparticles encapsulated into the microgels for the actuation and gathering of the microrobots in a targeted endovascular location under physiological blood flow, and an imaging agent such as tantalum microparticles for the accurate tracking of the microrobots inside a living organism by clinical medical equipment. Related systems and methods are disclosed, for delivering and guiding the microrobots to accumulate and aggregate in a targeted location, thereby providing an on-demand and reliable embolization therapy under real-time monitoring.

### CROSS-REFERENCE TO RELATED APPLICATIONS

The present application claims priority to U.S. Provisional Application No. 63/461,179, filed Apr. 21, 2023, and U.S. Provisional Application No. 63/533,103, filed Aug. 16, 2023, the full disclosures of which are incorporated herein by reference in their entirety for all purposes.

### BACKGROUND

Embolization therapy is a burgeoning minimally invasive endovascular procedure involving blocking blood vessels as a treatment for various diseases, such as tumors, fistulas, and arteriovenous malformations. Through deploying embolic devices (e.g., metallic coils) with the assistance of interventional catheterization technique, the vascular sac can be occupied and blood flow into the arteries can be excluded from circulation, thus preventing an aneurysm from continuously bulging to a point of rupture, or thus cutting off the nutrient supply to a tumor. Nevertheless, the application of embolization coils for the occlusion of targets that include larger vascular spaces, such as those of giant aneurysm and the false lumen of a dissected aorta, may not be practical because the procedure will involve a large number of coils and a prolonged procedure time and may yield incomplete embolization performance. Other embolic materials including proteinic sponges, hydrogel microspheres, and liquid agents have also been developed. The practical performance and long-term outcome when using these alternatives is still not satisfying, though, due to the occurrence of nonuniform filling effects, and more seriously, the distal occlusion of unexpected arteries caused by the leakage and fragmentation of embolic materials. Therefore, a new embolization strategy with better filling controllability, endovascular environment compatibility, and clinical performance is urgently desired.

The recent rise of microrobotics provides a revolutionary opportunity for embolization treatment. Due to their small size, active mobility, and remote controllability, microrobots that convert energy from surrounding environments into mechanical movement are able to access hard-to-reach lesion sites inside a human body dexterously and accurately, promising major benefits in a variety of biomedical fields. Therefore, employing active microrobots as embolic agents emerges as an inspiring embolization strategy to meet the expectation to fill a targeted vascular sac completely while avoiding unintentional blockage of nontargeted blood vessels.

However, existing microrobotic technologies cannot yet achieve this goal and several issues should be carefully considered in advance. For example, the effective and precise navigation of microrobots in complex and dynamic endovascular environments raises critical requirements for microrobotic control and imaging methodologies, especially in the vascular branches of the aorta, where physiological blood flow velocity can be as high as 10-20 cm/s. Further, it is crucial to the clinical feasibility of embolization therapy that, after they arrive at a targeted aneurysm, a vast number of microrobots can be noninvasively triggered to entirely occlude the sac without postoperative leakage and fragmentation. Moreover, the stability and compatibility of embolic microrobots with surrounding biological environments should be elaborately evaluated to guarantee the safety of their use and the long-term effects of a treatment they provide. The present disclosure addresses these and other issues by providing compositions and methods related to pH-responsive and magnetically guidable microrobots having several beneficial advantages for use in embolization therapies.

### BRIEF SUMMARY

The following summary provides a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. Covered embodiments of the disclosure are not defined by this summary. The subject matter should be understood by reference to appropriate portions of the entire specification, any or all figures, and each claim. Some of the exemplary embodiments of the present disclosure are discussed below.

The present disclosure generally relates to an interventional catheterization-integra ted swarming microrobotic platform. The platform is particularly useful for real-time medical imaging-guided embolization of aneurysms and blood vessels in a subject. Each building block of the microrobotic swarm, serving as the embolic agent, is a micro-sized sphere composed of a pH-responsive self-healing hydrogel matrix, magnetic nanoparticles, and imaging dopants. The disclosed microrobotic embolization strategy can be initiated by catheter-assisted delivery and deployment of swarming microgels to, e.g., an endovascular position, providing a highway across biological barriers and protecting the microrobots from the immune system inside a living body. A programmed or otherwise controllable external magnetic field can be subsequently applied to navigate and concentrate the swarming microrobots into, for example, a targeted sac. The application of the magnetic field can be assisted by real-time guidance of ultrasound and fluoroscopy imaging. An acidic buffer solution can be injected via catheter to activate the self-healing behavior of microgels, followed by the removal of catheter, which triggers the formation of inter/intramolecular hydrogen bonds among the hydrogel matrix and thus makes the microrobots spontaneously adhere with each other.

In one aspect, the disclosure relates to a pH-responsive self-healing microgel. The microgel includes a polymerization product of two or more polymer precursors, and a cross-linker. The microgel further includes a magnetic agent and an imaging agent.

In another aspect, the disclosure relates to a population of pH-responsive self-healing microgels. The population includes a pH-responsive self-healing microgel as described herein.

In another aspect, the disclosure relates to a system for treating a subject in need of an embolization at a targeted endovascular location. The system includes a population of pH-responsive self-healing microgels as described herein. The system further includes a magnetic control module, and an imaging module.

In another aspect, the disclosure relates to a method of treating a subject in need of an embolization at a targeted endovascular location in the subject. The method includes administering to the subject a population of pH-responsive self-healing microgels as described herein. The method further includes applying a magnetic field to at least a portion of the subject. The method further includes guiding the population of pH-responsive self-healing microgels to the targeted endovascular location using the magnetic field. The method further includes introducing an amount of an acidic solution to the targeted endovascular location. The amount is effective in inducing formation of hydrogen bonds between pH-responsive self-healing microgels of the population of pH-responsive self-healing microgels.

### BRIEF DESCRIPTION OF THE DRAWINGS

Illustrative aspects of the present disclosure are described in detail below with reference to the following drawing figures. It is intended that embodiments and figures disclosed herein are to be considered illustrative rather than restrictive. Like reference symbols and designations in the various drawings indicate like elements.

FIG. 1 presents a schematic illustration of a catheter-assisted microrobotic platform for on-demand embolization of aneurysm and artery in accordance with a provided embodiment.

FIG. 2 illustrates an exemplary setup of a microrobotic platform for on-demand embolization of aneurysm and artery in accordance with a provided embodiment.

FIG. 3 illustrates a workflow for preparation of embolic microrobots in accordance with a provided embodiment.

FIG. 4 illustrates a pH-responsive self-adhesive mechanism of embolic microrobots in accordance with a provided embodiment.

FIG. 5 presents images demonstrating the self-healing process of the provided hydrogels under biological conditions.

FIG. 6 presents results from a mechanical tensile test evaluating the healing strength of hydrogels under different conditions, and the effect of healing time on the fracture stress of healed hydrogels.

FIG. 7 presents graphs plotting the FTIR-ATR and Raman spectra of provided hydrogels during a healing process, with highlighted regions of the graphs indicating the formation of two hydrogen bonding types, i.e., the interleaved interaction between carboxyl and amide groups and the face-on interaction between opposite carboxyl groups.

FIG. 8 presents an optical image of microgels prepared according to a provided embodiment, and a graph plotting a size distribution of the prepared microgels.

FIG. 9 presents images showing spontaneous adhesion between provided microgels in saline and blood when an acidic buffer (pH: 4.5-5) is added.

FIG. 10 presents images and graphs showing the temporal shape variation of a provided disc-shaped embolic material in anti-coagulated porcine blood, the effect of blood incubation time on the area and compression modulus of disc-shaped embolic material, and the hemolysis analysis of red blood cells after incubation with different concentrations of provided microgels for 24 h.

FIG. 11 presents images showing the coagulation behavior of blood incubated with a provided disc-shaped embolic material.

FIG. 12 presents graphs showing the effect of microgel concentration on the viability of stem cells, 3T3 cells and HUVECs after 48 h of co-culture.

FIG. 13 presents images of a clinical catheter used for deployment of microgels in accordance with a provided embodiment.

FIG. 14 presents images showing the effect of Ta concentration on the imaging contrast of provided microgels under fluoroscopy.

FIG. 15 presents a schematic illustration of aneurysm embolization procedures in a dynamic fluid in accordance with a provided embodiment, including targeted catheterization (I), deployment and active accumulation of swarming microgels into an aneurysm sac under the actuation of robotic magnet (II), on-demand embolization via mild acid stimulus (III), and the removal of the catheter and robotic magnet (IV). Also presented are images showing experimental results of using the provided procedures. Additional images show the morphology of welded microgels after incubation at 37° C. for 6 months.

FIG. 16 presents images showing real-time ultrasound imaging of a provided aneurysm embolization process in a dynamic blood environment with an inlet mean velocity of 20 cm/s. Two imaging planes (I and II) are adopted to reflect the embolization conditions on horizontal and vertical cross sections of the aneurysm sac, respectively. The area surrounded by the dashed line of the images indicates the targeted aggregation of microgels. The disappearance of ultrasound Doppler signals inside the aneurysm sac demonstrates that the blood flow into the aneurysm is excluded from the circulation.

FIG. 17 presents images of an experimental setup for aneurysm replica embolization in blood under the real-time guidance of c-arm fluoroscopy. The setup includes a saccular aneurysm replica, a robotic magnet for magnetic swarm control, a peristaltic pump for blood flow control, a catheter controller for microrobot deployment, and a c-arm fluoroscopy system.

FIG. 18 illustrates real-time fluoroscopy imaging of an aneurysm embolization process in accordance with a provided embodiment.

FIG. 19 presents images showing the filling ratio of an aneurysm replica under different magnetic field parameters and flowing conditions. The inset images demonstrate the specific embolization performance in different regions. The rotating frequency of the magnetic field is fixed to 2 Hz.

FIG. 20 presents a schematic illustration and an image of an experimental setup for artery embolization in ex vivo human placenta.

FIG. 21 presents real-time ultrasound images of a blood vessel embolization process inside human placenta in accordance with a provided embodiment.

FIG. 22 presents images used for evaluating artery embolization performance via ultrasound Doppler signals.

FIG. 23 presents real-time fluoroscopy images of a blood vessel embolization process in accordance with a provided embodiment.

FIG. 24 presents images showing a blockage welded by self-adhesive microgels after on-demand artery embolization in accordance with a provided embodiment.

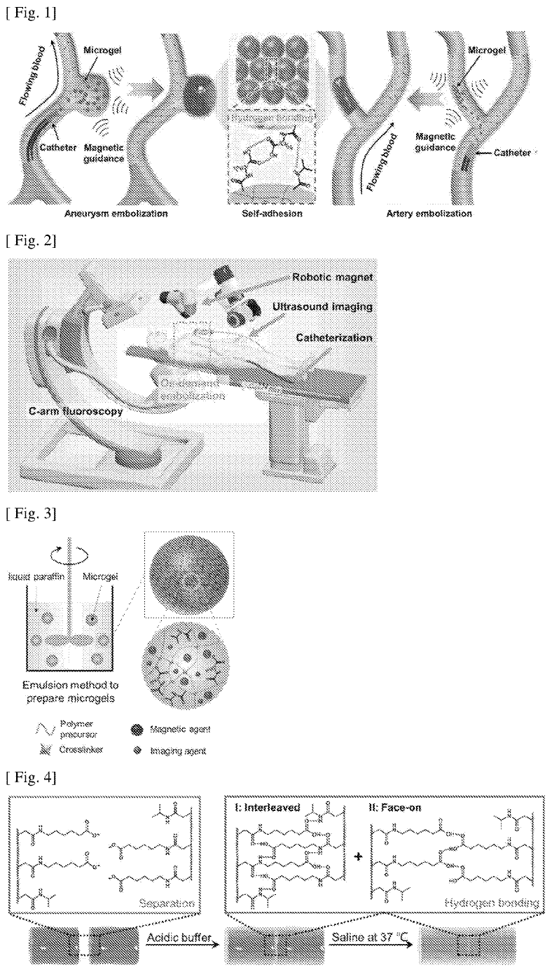

### DETAILED DESCRIPTION

### I. General

The present disclosure may be understood more easily by reference to the following detailed description and examples. The disclosure is not limited to the specific materials, devices, methods, or systems described and/or shown herein. Unless otherwise defined, all technical and scientific terms used herein have the same meaning as commonly understood by one of ordinary skill in the art to which the disclosure belongs. Although materials, devices, methods, and systems similar or equivalent to those described herein can be used in the practice or testing of the present invention, suitable methods and materials are described below.

The present disclosure provides a multifunctional microgel as a microrobot, and a resultant embolization system based on catheter and clinical imaging equipment. The particular composition of the provided hydrogel matrix for microrobots is carefully designed to advantageously satisfy requirements of on-demand embolization. For example, polymer precursors and crosslinkers were specifically selected to obtain the provided functional hydrogel material exhibiting several beneficial properties. Also advantageously, synthesis of the provided embolic microrobots can be achieved via a facile emulsion method. Generally, a self-healing hydrogel precursor containing a thermal initiator is first mixed with appropriate amounts of a magnetic agent and an imaging agent to obtain a homogeneous aqueous phase, which is then emulsified via a liquid paraffin (oil phase). Subsequently, the mixture is heated to trigger a polymerization reaction of the hydrogel precursor, thus solidifying the microemulsion into multifunctional embolic microrobots.

The disclosed materials and methods have been demonstrated to provide efficient and selective accumulation of microgels in an aneurysm model, achieving a filling ratio greater than 95% in even a dynamic blood flowing condition with a mean velocity of, for example, as high as 20 cm/s. Effective embolization and blood occlusion according to provided embodiments have been verified via ultrasound Doppler and X-ray imaging. Through careful modulation of polymeric chains of the hydrogel, the welding of microrobots can exhibit excellent self-adhesive and physical stability in a physiological environment for at least half a year, as well as satisfactory bio- and hemo-compatibility. Moreover, provided embodiments have realized the blockage of blood vessel in an ex vivo human placenta, validating the applicability of the disclosed embolization strategy for real vascular systems. The disclosure thus presents an integrated microrobotic platform with effective targeting and on-demand embolization capabilities, representing a substantial leap forward in the field of minimally invasive embolization treatments.

### II. Definitions

Unless defined otherwise, all technical and scientific terms used herein have the same meaning as commonly understood by one of ordinary skill in the art to which this disclosure belongs.

As used herein, the terms “fill ratio” and “filling ratio” refer to the percentage of the volume of an endovascular location that is occupied by an aggregation of the microgels disclosed herein.

As used herein, the term “heteroalkyl,” by itself or as part of another substituent, refers to an alkyl group of any suitable length and having from 1 to 3 heteroatoms such as N, O and S. Additional heteroatoms may also be useful, including, but not limited to, B, Al, Si and P. The heteroatoms may also be oxidized, such as, but not limited to, —S(O)— and —S(O)₂—. For example, heteroalkyl may include ethers, thioethers and alkyl-amines. The heteroatom portion of the heteroalkyl may replace a hydrogen of the alkyl group to form a hydroxy, thio, or amino group. Alternatively, the heteroatom portion may be the connecting atom, or be inserted between two carbon atoms. Heteroalkyl groups may be substituted or unsubstituted.

As used herein, the term “alkyl,” by itself or as part of another substituent, refers to a straight or branched, saturated, aliphatic radical having the number of carbon atoms indicated. A branched alkyl may include one or branches having a geminal, vicinal, and/or isolated pattern. For example, an alkyl may include gem-methyl groups. Alkyl may include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl may also refer to alkyl groups having up to 20 carbons atoms, such as, but not limited to heptyl, octyl, nonyl, decyl, etc. Alkyl groups may be substituted or unsubstituted. Unless otherwise specified, “substituted alkyl” groups may be substituted with one or more groups selected from halo, hydroxy, amino, alkylamino, amido, acyl, nitro, cyano, and alkoxy.

As used herein, the term “hydrogel” refers to a highly-interdependent, biphasic matrix comprising a solid component (usually a polymer, and more commonly a highly cross-linked polymer) that has both hydrophilic and hydrophobic character, and a liquid dispersion medium (e.g., water) that is retained in the matrix by intermolecular forces. The hydrophobic character provides the matrix with a degree of water insolubility while the hydrophilic character affords water permeability. One of skill in the art will appreciate that several different types of polymers may be used in combination to form hydrogels useful in the methods of the present invention. Being polymer networks that have high water-absorbing capacity, hydrogels often closely mimic native extracellular matrices. Hydrogels also tend to possess a degree of flexibility very similar to natural tissues, due to the relatively high water content. In some cases, hydrogels may contain well over 90% water.

As used herein, the term “polymer” refers to an organic substance composed of a plurality of repeating structural units (monomeric units) covalently linked to one another.

As used herein, the term “microgel” refers to a hydrogel structure having an equivalent spherical diameter of 10 nm-1 mm, that is, in the nanometer and micrometer ranges.

As used herein, the term “equivalent spherical diameter” refers to a property of a three-dimensional object indicating the greatest maximum diameter of any cross-section of the object that includes the geometric center of the object. The “maximum diameter” of a two-dimensional figure, e.g., a cross-section of a three-dimensional object, is the longest distance between any two points on the boundary of the figure. The “geometric center” of a three-dimensional object is the arithmetic mean of all points on the surface of the object.

As used herein, the term “substantially spherical” refers to a characterization of an object as one for which any cross-section of the object that includes the geometric center of the object has a maximum diameter within 50% of the equivalent spherical diameter of the object. Substantially spherical objects can also include objects for which any discrepancies between such maximum diameters and the equivalent spherical diameter are less than 35%, e.g., less than 25%, less than 15%, less than 10%, less than 7%, less than 5%, less than 4%, less than 3%, less than 2%, or less than 1% of the equivalent spherical diameter.

A hydrogel, microgel, or polymer is “pH-responsive” if the chemical structure of the gel or polymer changes in response to a change in the pH of the gel environment. A pH-responsive gel or polymer can be one in which chemical bonds, e.g., dynamic bonds, form or break in response to an environmental pH change.

A hydrogel, microgel, or polymer is “self-healing” or “self-adhesive” if dynamic bonds form across an interface of the gel or polymer, causing gel or polymer portions on opposite sides of the interface to adhere to one another, resulting in an interfacial welding of two or more surfaces. A self-healing gel or polymer thus can totally or partially recover its structure and properties after suffering damage, thereby recovering its physical integrity totally or partially. A self-healing gel or polymer can also be used to combine two or more previously separate structures of the gel or polymer, thereby forming a larger combined structure. This self-healing occurs due the presence of dynamic bonds that can recover by themselves after being broken.

As used herein, the term “imaging agent” refers to an agent comprising a detectable material useful for producing an image of a volume of interest, and in particular an image including more visual information than could otherwise be included in the absence of the imaging agent. The imaging agent can, for example, be a material that is incorporated by a substance within the volume to be imaged, that binds to a substance within the volume to be imaged, or that otherwise accumulates within the volume to be imaged.

An imaging agent that is a “contrast agent” is an imaging agent that comprise a material that can significantly attenuate incident radiation, e.g., X-ray radiation, causing a reduction of the radiation transmitted through the volume of interest. After undergoing image reconstruction and typical post-processing, this increased attenuation, e.g., X-ray attenuation, is interpreted as an increase in the density of the volume of interest, which creates a contrast enhancement in the volume comprising the contrast agent relative to the background, e.g., background tissue, in the image.

As used herein, the term “magnetic agent” refers to an agent comprising a magnetic material, e.g., a ferromagnetic, ferrimagnetic, or superparamagnetic material. These materials have a strong susceptibility to magnetization. Magnetic materials can become magnetized when a magnetic field is applied to the materials, and the strength of magnetization can depend on the strength of the magnetic field. In some such materials, magnetization can persist for a time period after removal of the magnetic field. Magnetic materials may not be permanent magnets, but rather can include materials like iron, which can be magnetized by a magnetic field from a permanent magnet or an electromagnet. Magnetic materials can be attracted to permanent magnets, but may not exert any magnetic attractive force on their own.

As used herein, the term “subject” refers to a vertebrate, and preferably to a mammal. Mammalian subjects for which the provided composition is suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age.

As used herein, the term “treatment” refers to an approach for obtaining beneficial or desired results including but not limited to a therapeutic benefit and/or a prophylactic benefit. A therapeutic benefit imparts any relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested. A treatment can involve any of ameliorating one or more symptoms of disease, e.g., cancer, preventing the manifestation of such symptoms before they occur; slowing down or completely preventing the progression of the disease (as may be evident by longer periods between reoccurrence episodes, slowing down or prevention of the deterioration of symptoms, etc.), enhancing the onset of a remission period, slowing down the irreversible damage caused in the progressive-chronic stage of the disease (both in the primary and secondary stages), delaying the onset of said progressive stage, or any combination thereof.

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the singular forms “a,” “an,” and “the” include plural referents unless the content clearly dictates otherwise. Thus, for example, reference to “apolymer” optionally includes a combination of two or more polymers, and the like.

As used herein, the term “about” denotes a range of value that is +/−10% of a specified value. For instance, “about 10” denotes the value range of 9 to 11 (10+/−1).

### III. Microgels

In one aspect, the present disclosure provides various pH-responsive self-healing microgels that generally include a polymerization reaction product of two or more polymer precursors and a cross-linker. The microgels generally further include one or more magnetic agents, and one or more imaging agents. In some embodiments, the microgel consists of the polymerization reaction product, the one or more magnetic agents, and the one or more imaging agents. In some embodiments, the microgel consists essentially of the polymerization reaction product, the one or more magnetic agents, and the one or more imaging agents. The particular combination and relative amounts of these components provide the microgel with several surprising improvements in various important characteristics. These improved characteristics are particularly advantageous when a population of the provided pH-responsive self-healing microgels are used, e.g., as a swarm of remotely controllable and detectable microrobots, in medical applications, and particularly when used in embolization therapies.

The polymerization reaction product of the provided pH-responsive self-healing microgel is the product of two or more polymer precursors and a cross-linker. The number of different species of polymer precursors used to form the polymerization reaction product can be, for example, three, four, five, six, seven, eight, nine, ten, or more than ten. The number of different species of cross-linker used to form the polymerization reaction product can be, for example, three, four, five, six, seven, eight, nine, ten, or more than ten. In some embodiments, the microgel includes only one polymerization reaction product. In some embodiments, the microgel includes two or more different species of polymerization reaction products, e.g., three or more, four or more, five or more, six or more, seven or more, eight or more, nine or more, or ten or more different species of polymerization reaction products. The compositions and relative amounts of the one or more polymerization reaction products of the microgel can be selected or configured to provide the microgel with its beneficial properties, including its pH-responsive and self-healing characteristics.

In some embodiments, at least one of the two or more polymer precursors of the polymerization reaction product includes one or both of an acryloyl group and an acrylamide group. The use of polymer precursors with acryloyl and/or acrylamide groups can provide the polymerization reaction product with desirable characteristics including, for example, high water solubility, and good biocompatability. In some embodiments, each of the two or more polymer precursors includes one or both of an acryloyl group and an acrylamide group. In some embodiments, at least one of the two or more polymer precursors of the polymerization reaction product includes an acryloyl group. In some embodiments, each of the two or more polymer precursors includes an acryloyl group. In some embodiments, at least one of the two or more polymer precursors of the polymerization reaction product includes an acrylamide group. In some embodiments, each of the two or more polymer precursors includes an acrylamide group. In some embodiments, at least one of the two or more polymer precursors of the polymerization reaction product includes an acryloyl group and an acrylamide group. In some embodiments, each of the two or more polymer precursors includes an acryloyl group and an acrylamide group.

The acryloyl group of the polymer precursor of the polymerization reaction product can include, for example, N-acryloyl 2-glycine, substituted N-acryloyl 2-glycine, N-acryloyl 4-aminobutyric acid, substituted N-acryloyl 4-aminobutyric acid, N-acryloyl 6-aminocaproic acid, substituted N-acryloyl 6-aminocaproic acid, N-acryloyl 8-aminocaprylic acid, substituted N-acryloyl 8-aminocaprylic acid, N-acryloyl 11-aminoundecanoic acid, substituted N-acryloyl 11-aminoundecanoic acid, or any combination thereof. The acrylamide group of the polymer precursor of the polymerization reaction product can include, for example, N-isopropylacrylamide, substituted N-isopropylacrylamide, acrylamide, substituted acrylamide, acrylic acid, substituted acrylic acid, or any combination thereof.

In some embodiments, the polymerization reaction product of the provided microgel includes a plurality of pendant side chains. In some embodiments, each pendant side chain of at least a portion of the pendant side chains independently includes an amide group. In some embodiments, each pendant side chain of the polymerization reaction product independently includes an amide group. In some embodiments, each pendant side chain of at least a portion of the pendant side chains independently includes a carboxyl group. In some embodiments, each pendant side chain of the polymerization reaction product independently includes a carboxyl group. In some embodiments, each pendant side chain of at least a portion of the pendant side chains independently includes an amide group and a carboxyl group. In some embodiments, each pendant side chain of the polymerization reaction product independently includes an amide group and a carboxyl group. The use of a polymerization reaction product with amide and/or carboxyl groups in its pendant side chains can provide the polymerization reaction product with desirable characteristics including, for example, enhanced water solubility, and the ability to form hydrogen bonds, e.g., hydrogen bonds providing the microgel with its pH-responsive and self-healing properties. The polymerization reaction product can include only one species of pendant side chains, or can include two or more different species of pendant side chains, e.g., three or more, four or more, five or more, six or more, seven or more, eight or more, nine or more, or ten more different species of pendant side chains.

In some embodiments, each pendant side chain of at least a portion of the pendant side chains independently includes or consists of an optionally oxo-substituted 3- to 12-membered heteroalkyl. In some embodiments, each pendant side chain of the polymerization reaction product independently includes or consists of an optionally oxo-substituted 3- to 12-membered heteroalkyl. In some embodiments, each pendant side chain of at least a portion of the pendant side chains has the structure of the formula:

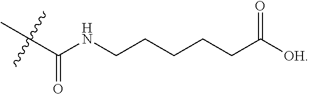 [SMILES: *C(=O)NCCCCCC(=O)O]

In some embodiments, each pendant side of the polymerization reaction product has the structure of the above formula.

In some embodiments, the plurality of the pendant side chains of the polymerization reaction product can form hydrogen bonds with one another. The hydrogen bonds can include, for example, a hydrogen bond between a carboxyl group of one pendant side chain, and a carboxyl group of a different pendant side chain, e.g., a pendant side chain across an interface of the microgel, and/or a pendant side chain of a different microgel. The hydrogen bonds can additionally or alternatively include a hydrogen bond between a carboxyl group of one pendant side chain, and an amide group of a different pendant side chain, e.g., a pendant side chain across an interface of the microgel, a pendant side chain of a different microgel. The hydrogen bonds can include or consist of hydrogen bonds between terminal function groups, e.g., terminal carboxyl groups, of different pendant side chains. Such hydrogen bonds are referred to herein as “face-on” hydrogen bonds. The hydrogen bonds can alternatively or additionally include or consist of hydrogen bonds between terminal functional groups and mid-chain functional groups of different pendant side chains. Such hydrogen bonds are referred to herein as “interleaved” hydrogen bonds. The ability of the polymerization reaction product to form a combination of face-on and interleaved hydrogen bonds can advantageously provide the microgel with its self-healing properties.

In some embodiments, the polymerization reaction product of the provided microgel forms hydrogen bonds when the microgel is exposed to an acidic environment having a pH below a certain threshold. This characteristic of the polymerization reaction product can provide the microgel with its advantageous pH-responsive properties. The polymerization reaction product can be selected or configured to form hydrogen bonds when the microgel is exposed to a pH that is, for example, less than about 6, e.g., less than about 5.75, less than about 5.5, less than about 5.25, less than about 5, less than about 4.75, less than about 4.5, less than about 4.25, less than about 4, or less than about 3.75. The threshold pH triggering the formation of hydrogen bonds among pendant side chains of the polymerization reaction product can be, for example, between about 3.5 and about 6, e.g., between about 3.5 and about 5, between about 3.75 and about 5.25, between about 4 and about 5.5, between about 4.25 and about 5.75, or between about 4.5 and about 6. Beneficially, the hydrogen bonds, once formed, can be stable under typical physiological conditions. For example, the formed hydrogen bonds of the polymerization reaction product can be maintained in 0.9 wt % saline at 37° C. for at least about 8 hours, e.g., at least about 10 hours, at least about 12 hours, at least about 15 hours, at least about 19 hours, at least about 24 hours, at least about 30 hours, at least about 37 hours, at least about 46 hours, at least about 58 hours, or at least about 72 hours.

The cross-linker used to form the polymerization reaction product of the provided microgel can be selected to provide the microgel with a desired beneficial mechanical strength. In some embodiments, at least one cross-linker of the polymerization reaction product includes an acrylamide group. In some embodiments, each cross-linker of the polymerization reaction product independently includes an acrylamide group. In some embodiments, at least one cross-linker of the polymerization reaction product includes an acrylate group. In some embodiments, each cross-linker of the polymerization reaction product independently includes an acrylate group. In some embodiments, at least one cross-linker of the polymerization reaction product includes a methacrylate group. In some embodiments, each cross-linker of the polymerization reaction product independently includes a methacrylate group. In some embodiments, at least one cross-linker of the polymerization reaction product includes a vinyl ether group. In some embodiments, each cross-linker of the polymerization reaction product independently includes a vinyl ether group. Cross-linkers suitable for forming the polymerization reaction product include, for example, N,N′-methylenebisacrylamide, substituted N,N′-methylenebisacrylamide, poly(ethylene glycol) diacrylate, substituted poly(ethylene glycol) diacrylate, 1,4-cyclohexanedimethanol divinyl ether, substituted 1, 4-cyclohexanedimethanol divinyl ether, trimethylolpropane triacrylate, substituted trimethylolpropane triacrylate, methacrylated gelatin, substituted methacrylated gelatin, and any combination thereof.

The amount of the polymerization reaction product components within the provided microgel can be selected to provide the microgel with its beneficial pH-responsive and self-healing properties, and to enhance other desirable characteristics of the microgel including, for example, aqueous solubility, biocompatibility, and mechanical strength. The combined concentration of the two or more polymer precursors in the microgel (i.e., the concentration of polymer precursors as present in the form of a component of the polymerization reaction product in the microgel) can be, for example, between about 10 wt % and about 30 wt %, e.g., between about 10 wt % and about 22 wt %, between about 12 wt % and about 24 wt %, between about 14 wt % and about 26 wt %, between about 16 wt % and about 28 wt %, or between about 18 wt % and about 30 wt %. In terms of upper limits, the combined concentration of the polymer precursors in the microgel can be, for example, less than about 30 wt %, e.g., less than about 28 wt %, less than about 26 wt %, less than about 24 wt %, less than about 22 wt %, less than about 20 wt %, less than about 18 wt %, less than about 16 wt %, less than about 18 wt %, less than about 16 wt %, less than about 14 wt %, or less than about 12 wt %. In terms of lower limits, the combined concentration of the polymer precursors in the microgel can be, for example, greater than about 10 wt %, e.g., greater than about 12 wt %, greater than about 14 wt %, greater than about 16 wt %, greater than about 18 wt %, greater than about 20 wt %, greater than about 22 wt %, greater than about 24 wt %, greater than about 26 wt %, or greater than about 28 wt %. Higher polymer cursor concentrations, e.g., greater than about 30 wt %, and lower polymer precursor concentrations, e.g., less than about 10 wt %, are also contemplated.

The concentration of the cross-linker in the provided pH-responsive self-healing microgel (i.e., the concentration of cross-linker as present in the form of a component of the polymerization reaction product in the microgel) can be, for example, between about 0.1 wt % and about 1 wt %, e.g., between about 0.1 wt % and about 0.64 wt %, between about 0.19 wt % and about 0.73 wt %, between about 0.28 wt % and about 0.82 wt %, between about 0.37 wt % and about 0.91 wt %, or between about 0.46 wt % and about 1 wt %. In terms of upper limits, the cross-linker concentration in the microgel can be, for example, less than about 1 wt %, e.g., less than about 0.91 wt %, less than about 0.82 wt %, less than about 0.73 wt %, less than about 0.64 wt %, less than about 0.55 wt %, less than about 0.46 wt %, less than about 0.37 wt %, less than about 0.28 wt %, or less than about 0.19 wt %. In terms of lower limits, the cross-linker concentration in the microgel can be, for example, greater than about 0.1 wt %, e.g., greater than about 0.19 wt %, greater than about 0.28 wt %, greater than about 0.37 wt %, greater than about 0.46 wt %, greater than about 0.55 wt %, greater than about 0.64 wt %, greater than about 0.73 wt %, greater than about 0.82 wt %, or greater than about 0.91 wt %. Higher cross-linker concentrations, e.g., greater than about 1 wt %, and lower cross-linker concentrations, e.g., less than about 0.1 wt %, are also contemplated.

In some embodiments, the magnetic agent of the provided pH-responsive self-healing gel includes one or more transition metals, e.g., two or more, three or more, four or more, five or more, six or more, seven or more, eight or more, nine or more, or ten or more transition metals. Transition metals suitable for inclusion in the magnetic agent of the microgel include, for example, iron, nickel, cobalt, scandium, titanium, vanadium, chromium, manganese, copper, zinc, yttrium, zirconium, niobium, molybdenum, technetium, ruthenium, rhodium, palladium, gold, cadmium, lanthanum, hafnium, tantalum, tungsten, rhenium, osmium, iridium, platinum, silver, actinium, and any combination thereof. In some embodiments, the magnetic agent includes iron, nickel, cobalt, or a combination thereof. In some embodiments, the magnetic agent includes or consists of a nickel microparticle. In some embodiments, the magnetic agent includes or consists of an iron microparticle. In some embodiments, the magnetic agent includes or consists of an iron nanoparticle. In some embodiments, the magnetic agent includes or consists of an iron oxide nanoparticle. In some embodiments, the magnetic agent includes or consists of a cobalt oxide nanoparticle. In some embodiments, the magnetic agent includes or consists of an iron-platinum nanoparticle. The magnetic agent can, for example, include or consist of one or more magnetite nanoparticles.

The amount of the magnetic agent within the provided pH-responsive self-healing microgel can be selected to be high enough to advantageously allow the microgel to be effectively and efficiently remotely guided, for example with an applied magnetic field, while also being low enough to not interfere with other beneficial microgel characteristics, such as biocompatibility and mechanical properties. The concentration of the magnetic agent in the microgel can be, for example, between about 10 wt % and about 30 wt %, e.g., between about 10 wt % and about 22 wt %, between about 12 wt % and about 24 wt %, between about 14 wt % and about 26 wt %, between about 16 wt % and about 28 wt %, or between about 18 wt % and about 30 wt %. In terms of upper limits, the concentration of the magnetic agent in the microgel can be, for example, less than about 30 wt %, e.g., less than about 28 wt %, less than about 26 wt %, less than about 24 wt %, less than about 22 wt %, less than about 20 wt %, less than about 18 wt %, less than about 16 wt %, less than about 14 wt %, or less than about 12 wt %. In terms of lower limits, the concentration of the magnetic agent in the microgel can be, for example, greater than about 10 wt %, e.g., greater than about 12 wt %, greater than about 14 wt %, greater than about 16 wt %, greater than about 18 wt %, greater than about 20 wt %, greater than about 22 wt %, greater than about 24 wt %, greater than about 26 wt %, or greater than about 28 wt %. Higher magnetic agent concentrations, e.g., greater than about 30 wt %, and lower magnetic agent concentrations, e.g., less than about 10 wt %, are also contemplated.

In some embodiments, the imaging agent of the provided pH-responsive self-healing microgel includes or consists of one or more contrast agents. Contrast agents suitable for use with the microgel include, for example, barium sulfate microparticles, tantalum microparticles, iodipin, and any combination thereof. In some embodiments, the imaging agent of the microgel includes or consists of one or more tantalum microparticles.

The amount of the imaging agent within the provided pH-responsive self-healing microgel can be selected to be high enough to advantageously allow the microgel to be effectively and efficiently imaged, for example when inside the body of a subject, while also being low enough to not interfere with other beneficial microgel characteristics, such as biocompatibility and mechanical properties. The concentration of the imaging agent in the microgel can be, for example, between about 5 wt % and about 15 wt %, e.g., between about 5 wt % and about 11 wt %, between about 6 wt % and about 12 wt %, between about 7 wt % and about 13 wt %, between about 8 wt % and about 14 wt %, or between about 9 wt % and about 15 wt %. In terms of upper limits, the concentration of the imaging agent in the microgel can be, for example, less than about 15 wt %, e.g., less than about 14 wt %, less than about 13 wt %, less than about 12 wt %, less than about 11 wt %, less than about 10 wt %, less than about 9 wt %, less than about 8 wt %, less than about 7 wt %, or less than about 6 wt %. In terms of lower limits, the concentration of the imaging agent in the microgel can be, for example, greater than about 5 wt %, e.g., greater than about 6 wt %, greater than about 7 wt %, greater than about 8 wt %, greater than about 9 wt %, greater than about 10 wt %, greater than about 11 wt %, greater than about 12 wt %, greater than about 13 wt %, or greater than about 14 wt %. Higher imaging agent concentrations, e.g., greater than about 15 wt %, and lower imaging agent concentrations, e.g., less than about 5 wt %, are also contemplated.

The particularly useful properties of the provided microgel have been demonstrated to be the result not only of the separate concentrations of individual components of the microgel, but also of the amounts of the components in relation to one another. Notably, the importance of the component ratios in simultaneously enabling different advantageous characteristics had not been previously appreciated. For example, certain relative amounts of the polymer precursors with respect to the cross-linker provide the microgel with its advantageous features. The mass ratio of the polymer precursor to the cross-linker within the microgel can be, for example, between about 10:1 and about 300:1, e.g., between about 10:1 and about 77:1, between about 14:1 and about 110:1 between about 20:1 and about 150:1, between about 28:1 and about 210:1, or between about 39:1 and about 300:1. In terms of upper limits, the mass ratio of the polymer precursor to the cross-linker within the microgel can be, for example, less than about 300:1, e.g., less than about 210:1, less than about 150:1, less than about 110:1, less than about 77:1, less than about 55:1, less than about 39:1, less than about 28:1, less than about 19:1, or less than about 14:1. In terms of lower limits, the mass ratio of the polymer precursor to the cross-linker within the microgel can be, for example, greater than about 10:1, e.g., greater than about 14:1, greater than about 20:1, greater than about 28:1, greater than about 39:1, greater than about 55:1, greater than about 77:1, greater than about 110:1, greater than about 150:1, or greater than about 210:1. Higher mass ratios, e.g., greater than about 300:1, and lower mass ratios, e.g., less than about 10:1, are also contemplated.

Certain relative amounts of the polymer precursors in the pH-responsive self-healing microgel to the magnetic agent in the microgel have also been demonstrated as providing the microgel disclosed herein with its surprisingly useful properties. The mass ratio of the polymer precursor to the magnetic agent within the microgel can be, for example, between about 0.3:1 and about 3.3:1, e.g., between about 0.3:1 and about 2.1:1, between about 0.6:1 and about 2.4:1, between about 0.9:1 and about 2.7:1, between about 1.2:1 and about 3:1, or between about 1.5:1 and about 3.3:1. In terms of upper limits, the mass ratio of the polymer precursor to the magnetic agent in the microgel can be, for example, less than about 3.3:1, e.g., less than about 3:1, less than about 2.7:1, less than about 2.4:1, less than about 2.1:1, less than about 1.8:1, less than about 1.5:1, less than about 1.2:1, less than about 0.9:1, or less than about 0.6:1. In terms of lower limits, the mass ratio of the polymer precursor to the magnetic agent in the microgel can be, for example, greater than about 0.3:1, e.g., greater than about 0.6:1, greater than about 0.9:1, greater than about 1.2:1, greater than about 1.5:1, greater than about 1.8:1, greater than about 2.1:1, greater than about 2.4:1, greater than about 2.7:1, or greater than about 3:1. Higher mass ratios, e.g., greater than about 3.3:1, and lower mass ratios, e.g., less than about 0.3:1, are also contemplated.

Certain relative amounts of the polymer precursors in the pH-responsive self-healing microgel to the imaging agent in the microgel have also been demonstrated as providing the microgel disclosed herein with its surprisingly useful properties. The mass ratio of the polymer precursor to the imaging agent within the microgel can be, for example, between about 0.6:1 and about 6.1:1, e.g., between about 0.6:1 and about 3.9:1, between about 1.15:1 and about 4.45:1, between about 1.7:1 and about 5:1, between about 2.25:1 and about 5.55:1, or between about 2.8:1 and about 6.1:1. In terms of upper limits, the mass ratio of the polymer precursor to the imaging agent in the microgel can be, for example, less than about 6.1:1, e.g., less than about 5.55:1, less than about 5:1, less than about 4.45:1, less than about 3.9:1, less than about 3.35:1, less than about 2.8:1, less than about 2.25:1, less than about 1.7:1, or less than about 1.15:1. In terms of lower limits, the mass ratio of the polymer precursor to the imaging agent in the microgel can be, for example, greater than about 0.6:1, e.g., greater than about 1.15:1, greater than about 1.7:1, greater than about 2.25:1, greater than about 2.8:1, greater than about 3.35:1, greater than about 3.9:1, greater than about 4.45:1, greater than about 5:1, or greater than about 5.55:1. Higher mass ratios, e.g., greater than about 6.1:1, and lower mass ratios, e.g., less than about 0.6:1, are also contemplated.

Certain relative amounts of the magnetic agent in the pH-responsive self-healing microgel to the cross-linker in the microgel have also been demonstrated as providing the microgel disclosed herein with its surprisingly useful properties. The mass ratio of the magnetic agent to the cross-linker within the microgel can be, for example, between about 10:1 and about 300:1, e.g., between about 10:1 and about 77:1, between about 14:1 and about 110:1 between about 20:1 and about 150:1, between about 28:1 and about 210:1, or between about 39:1 and about 300:1. In terms of upper limits, the mass ratio of the magnetic agent to the cross-linker in the microgel can be, for example, less than about 300:1, e.g., less than about 210:1, less than about 150:1, less than about 110:1, less than about 77:1, less than about 55:1, less than about 39:1, less than about 28:1, less than about 19:1, or less than about 14:1. In terms of lower limits, the mass ratio of the magnetic agent to the cross-linker in the microgel can be, for example, greater than about 10:1, e.g., greater than about 14:1, greater than about 20:1, greater than about 28:1, greater than about 39:1, greater than about 55:1, greater than about 77:1, greater than about 110:1, greater than about 150:1, or greater than about 210:1. Higher mass ratios, e.g., greater than about 300:1, and lower mass ratios, e.g., less than about 10:1, are also contemplated.

Certain relative amounts of the magnetic agent in the pH-responsive self-healing microgel to the imaging agent in the microgel have also been demonstrated as providing the microgel disclosed herein with its surprisingly useful properties. The mass ratio of the magnetic agent to the imaging agent within the microgel can be, for example, between about 0.6:1 and about 6.1:1, e.g., between about 0.6:1 and about 3.9:1, between about 1.15:1 and about 4.45:1, between about 1.7:1 and about 5:1, between about 2.25:1 and about 5.55:1, or between about 2.8:1 and about 6.1:1. In terms of upper limits, the mass ratio of the magnetic agent to the imaging agent in the microgel can be, for example, less than about 6.1:1, e.g., less than about 5.55:1, less than about 5:1, less than about 4.45:1, less than about 3.9:1, less than about 3.35:1, less than about 2.8:1, less than about 2.25:1, less than about 1.7:1, or less than about 1.15:1. In terms of lower limits, the mass ratio of the magnetic agent to the imaging agent in the microgel can be, for example, greater than about 0.6:1, e.g., greater than about 1.15:1, greater than about 1.7:1, greater than about 2.25:1, greater than about 2.8:1, greater than about 3.35:1, greater than about 3.9:1, greater than about 4.45:1, greater than about 5:1, or greater than about 5.55:1. Higher mass ratios, e.g., greater than about 6.1:1, and lower mass ratios, e.g., less than about 0.6:1, are also contemplated.

Certain relative amounts of the imaging agent in the pH-responsive self-healing microgel to the cross-linker in the microgel have also been demonstrated as providing the microgel disclosed herein with its surprisingly useful properties. The mass ratio of the imaging agent to the cross-linker within the microgel can be, for example, between about 5:1 and about 150:1, e.g., between about 5:1 and about 38:1, between about 7.1:1 and about 54:1, between about 9.9:1 and about 76:1, between about 14:1 and about 110:1, or between about 19:1 and about 150:1. In terms of upper limits, the mass ratio of the imaging agent to the cross-linker in the microgel can be, for example, less than about 150:1, e.g., less than about 110:1, less than about 76:1, less than about 54:1, less than about 38:1, less than about 27:1, less than about 19:1, less than about 14:1, less than about 9.9:1, or less than about 7:1. In terms of lower limits, the mass ratio of the imaging agent to the cross-linker in the microgel can be, for example, greater than about 5:1, e.g., greater than about 7:1, greater than about 9.9:1, greater than about 14:1, greater than about 19:1, greater than about 27:1, greater than about 38:1, greater than about 54:1, greater than about 76:1, or greater than about 110:1. Higher mass ratios, e.g., greater than about 150:1, and lower mass ratios, e.g., less than about 5:1, are also contemplated.

The size of the provided pH-responsive self-healing microgel can be configured or selected to have dimensions suitable for a particular desired application. For example, when the microgel is to be used in an embolization therapy, the microgel size can be configured or selected to facilitate the ability of the microgel to be delivered or guided to a targeted endovascular location, and the ability of the microgel to form an embolization, i.e., blockage, at that location. The microgel can have an equivalent spherical diameter that is, for example, between about 30 μm and about 300 μm, e.g., between about 30 μm and about 120 μm, between about 38 μm and about 150 μm, between about 48 μm and about 190 μm, between about 60 μm and about 240 μm, or between about 75 μm and about 300 μm. In terms of upper limits, the equivalent spherical diameter of the microgel can be, for example, less than about 300 μm, e.g., less than about 240 μm, less than about 190 μm, less than about 150 μm, less than about 120 μm, less than about 95 μm, less than about 75 μm, less than about 60 μm, less than about 48 μm, or less than about 38 μm. In terms of lower limits, the equivalent spherical diameter of the microgel can be, for example, greater than about 30 μm, e.g., greater than about 38 μm, greater than about 48 μm, greater than about 60 μm, greater than about 75 μm, greater than about 95 μm, greater than about 120 μm, greater than about 150 μm, greater than about 190 μm, or greater than about 240 μm. Larger diameters, e.g., greater than about 300 μm, and smaller diameters, e.g., less than about 30 μm, are also contemplated. In some embodiments, the microgel has a substantially spherical shape.

Also provided are populations of the pH-responsive self-healing microgels disclosed herein. The populations can include a plurality of microgels that are each members of the same provided species of microgels, i.e., that each have similar or identical compositions and properties. Alternatively, the populations can include a plurality of microgels that includes members of two or more different species of the provided microgels, i.e., microgels having different compositions and properties. The microgels of the provided population of microgels can have an average equivalent spherical diameter that is, for example, between about 30 μm and about 300 μm, e.g., between about 30 μm and about 120 μm, between about 38 μm and about 150 μm, between about 48 μm and about 190 μm, between about 60 μm and about 240 μm, or between about 75 μm and about 300 μm. In terms of upper limits, the average equivalent spherical diameter of the microgels of the population can be, for example, less than about 300 μm, e.g., less than about 240 μm, less than about 190 μm, less than about 150 μm, less than about 120 μm, less than about 95 μm, less than about 75 μm, less than about 60 μm, less than about 48 μm, or less than about 38 μm. In terms of lower limits, the average equivalent spherical diameter of the microgels of the population can be, for example, greater than about 30 μm, e.g., greater than about 38 μm, greater than about 48 μm, greater than about 60 μm, greater than about 75 μm, greater than about 95 μm, greater than about 120 μm, greater than about 150 μm, greater than about 190 μm, or greater than about 240 μm. Larger average diameters, e.g., greater than about 300 μm, and smaller average diameters, e.g., less than about 30 μm, are also contemplated.

### IV. Systems

Another aspect of the present disclosure relates to systems including a population of pH-responsive self-healing microgels. The microgels can be any of those disclosed in Section III. For example, the microgels of the provided system can each independently be a pH-responsive self-healing microgel that includes a magnetic agent, an imaging agent, and a polymerization reaction product of two or more polymer precursors and a cross-linker. The magnetic agent of the system microgels can include, for example, a transition metal-including nanoparticle, e.g., a magentite nanoparticle. The imaging agent of the system microgels can include, for example, a contrast agent, e.g., a tantalum nanoparticle. The polymerization reaction product of the system microgels can include, for example, a plurality of side chains including amide groups and/or carboxyl groups, e.g., 1-oxo-1-(5-carboxy) pentanaminomethyl side chains. Because the systems include a population of the particular microgels disclosed herein, the systems benefit from the advantageous characteristics described in Section III in relation to the microgels. Accordingly, the systems provide particular advantages when used to treat a subject in need of an embolization at a targeted endovascular location.

The provided systems generally further include a magnetic control module. The magnetic control module can be configured or selected to be capable of interacting with the magnetic agent of the population of pH-responsive self-healing microgels of the system, such that the magnetic control module can guide the microgel population, e.g., within the body of a subject in need of an embolization therapy. This remote control of the microgel population with the magnetic control module can be used to guide the microgel population, for example, to a targeted endovascular location. The magnetic control module can include, for example, a spherical permanent magnetic. Inclusion of a spherical permanent can beneficially allow the system to readily establish a localized magnetic field in an omnidirectional manner. The magnetic control module can include, for example, an electric motor and/or a robotic arm. These components of the magnetic control module can be, for example, configured to adjustably position the spherical permanent magnet in various locations relative to a subject for the purpose of guiding the microgel population within the subject.

The provided systems also generally include an imaging module. The imaging module can be configured or selected to be capable of detecting the imaging agent of the population of pH-responsive self-healing microgels of the system, such that the imaging module can provide positional information of the microgel population, e.g., information indicating the position of the microgel population within the body of a subject in need of embolization therapy. The imaging module can include or consist of a fluorescence microscopy module, e.g., a module configured or selected to be capable of detecting a fluorescent imaging agent of the microgel population. The imaging module can include or consist of a positron emission tomography module, e.g., a module configured or selected to be capable of detecting a radioactive imaging agent of the microgel population. The imaging module can include or consist of an ultrasound imaging what module, e.g., a module configured or selected to be capable of detecting an ultrasound contrast imaging agent of the microgel population. The imaging module can include or consist of a fluoroscopy module, e.g., a module configured or selected to be capable of detecting an X-ray contrast imaging agent of the microgel population. The imaging module can include or consist of a confocal microscopy module. In some embodiments, the imaging module of the system includes or consists of an ultrasound imaging module and a fluoroscopy module.

The provided systems can optionally further include a catheter. The catheter can be configured or selected to be capable of administering the population of microgels to a subject in need of an embolization therapy. The catheter can additionally or alternatively be configured or selected to be capable of administering an acidic solution, e.g., an acidic aqueous buffered solution, to the subject. thereby triggering pH-responsive self-healing properties of the microgels. The system can include a first catheter intended for administration of the microgel population, and a second catheter intended for administration of an acidic solution. The system can alternatively include a single catheter for administration of both the microgel population and the acidic solution.

The provided systems can optionally further include a computer system configured or selected for operating one or more other components of the systems, and/or for performing one or more steps of any of the methods disclosed herein. The computer system can include, for example, a logic system that receives one or more data signals from the magnetic control module and/or the imaging module, and/or that transmits one or more data signals to the magnetic control module and/or the imaging module. The logic system can include or be coupled with a display (e.g., monitor, LED display, etc.) and a user input device (e.g., mouse, keyboard, buttons, etc.). The logic system and the other components of the system can be part of a stand-alone or network connected computer system, or they can be directly attached to or incorporated in a device. The logic system can also include software that executes in a processor. The logic system can include a computer readable medium storing instructions for operating one or more other components of the systems, and/or for performing one or more steps of any of the methods disclosed herein.

Any of the computer systems provided herein can utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems can be interconnected via a system bus. Additional subsystems such as a printer, keyboard, storage device(s), monitor (e.g., a display screen, such as an LED), which is coupled to display adapter, and others can be included. Peripherals and input/output (I/O) devices, which couple to an I/O controller, can be connected to the computer system by any number of means known in the art such as an input/output (I/O) port (e.g., USB, FIREWIRE®). For example, an I/O port or external interface (e.g., Ethernet, Wi-Fi, etc.) can be used to connect the computer system to a wide area network such as the Internet, a mouse input device, or a scanner. The interconnection via the system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Additional subsystem can be data collection devices, such as a camera, microphone, accelerometer, and the like. Any of the data mentioned herein can be output from one component to another component and can be output to the user.

A computer system can include a plurality of the same components or subsystems, e.g., connected together by an external interface, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communications between devices, including at least 100, 200, 500, 1,000, 10,000, 50,000, 100,000, 500, 00, or one million communications. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium can be any combination of such devices. In addition, the order of operations may be re-arranged. A process can be terminated when its operations are completed, but could have additional steps not otherwise described herein. A process can correspond to a method, a function, a procedure, a subroutine, a subprogram, etc. When a process corresponds to a function, its termination can correspond to a return of the function to the calling function or the main function.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

### V. Methods of Treatment

Another aspect of the present disclosure relates to methods of treatment, e.g., methods for treating a subject in need of an embolization therapy. The methods generally include a step of administering a population of microgels to the subject, where the population can be any of those disclosed in Section IV. This administration can be via any suitable means. In particular embodiments, the microgel population is administered to the subject using a catheter. Because the treatment method thus uses the pH-responsive self-healing microgels of Section III, the method benefits from the related advantages described in that Section. For example, the method beneficially employs the microgel population as a swarm of remotely controllable and detectable microrobots, especially useful for embolization therapies.

The administration of the population of pH-responsive self-healing microgels to the subject can further include making an incision in the subject, and inserting a distal end of the catheter through the incision and into the subject. The administration can include anesthetizing a skin area of the subject prior to the making of the incision. The incision can be performed to access a blood vessel of the subject, for example, an artery, e.g., a femoral artery, such that the catheter can be inserted into the artery. A proximal end of the catheter can in some examples be loaded onto a pump, such as a syringe pump, where the pump is used to drive flow of the microgels through the catheter and into the subject.

The provided treatment methods generally further include applying a magnetic field to at least a portion of the subject, e.g., an area of the subject's body that is coinciding with, or proximate to, a location of the population of microgels after the population has been administered to the subject. The methods also generally include a step of guiding the microgel population within the subject using the applied magnetic field. Typically, the method involves guiding the microgel population to a an endovascular location in the subject, where the endovascular location is a target site for embolization with the microgel population. As one example, the targeted endovascular location can be within, adjacent to, or proximate to an aneurysm of the subject. The applying and guiding of the magnetic field can be performed, for example, using a magnetic control module. The magnetic control module can be, for example, any of those described in Section IV. For example, the magnetic control module can include a spherical permanent magnet, an electric motor, and/or a robotic arm.

The provided treatment methods can optionally include an operation of determining positional information for the microgel population within the subject by imaging the population in the subject. This positional information can advantageously be used during the guiding of the microgel population with the applied magnetic field. In particularly useful examples of the provided methods, the imaging used to determining the positional information includes real-time imaging, thereby allowing the guiding of the microgel population to be performed with knowledge of the current population location. The imaging of the population within the subject can be performed, for example, using an imaging module. The imaging module can be, for example, any of those described in Section IV. For example, the imaging module can be one configured or selected to, for example, provide ultrasound imaging and fluoroscopy imaging of the microgel population.

The provided treatment methods generally further include an operation of introducing an acidic solution to the subject, e.g., to a targeted endovascular location within the subject. Typically, the acidic solution is administered to the subject after the population of microgels has been guided to the targeted endovascular location. By changing the pH environment of the microgel population, the administered acidic solution induces formation of hydrogen bonds in the population. This hydrogen bond formation, which as described in Section III is an advantageous pH-responsive self-healing feature of the provided microgels, results in different microgels of the population adhering to one another, thereby therapeutically forming an aggregate, i.e., blockage, that can substantially fill the targeted endovascular location. The hydrogen bonds formed during the method can include face-on hydrogen bonds between different polymer side chains of the microgels, interleaved hydrogen bonds between different polymer side chains of the microgels, or a combination thereof. The introducing of the acidic solution can be performed by any suitable means. In particular embodiments, the acidic solution is introduced using a catheter. The catheter can be the same catheter also used to administer the population of microgels, or can be a different catheter. The acidic solution can be, for example, a buffered solution, an aqueous solution, or a buffered aqueous solution.

The amount and pH of the acidic solution introduced in the provided treatment method can be selected to be effective in inducing formation of hydrogen bonds between different microgels of the population of microgels. The pH of the acidic solution can be, for example, less than about 6, e.g., less than about 5.75, less than about 5.5, less than about 5.25, less than about 5, less than about 4.75, less than about 4.5, less than about 4.25, less than about 4, or less than about 3.75. The pH of the acidic solution can be, for example, between about 3.5 and about 6, e.g., between about 3.5 and about 5, between about 3.75 and about 5.25, between about 4 and about 5.5, between about 4.25 and about 5.75, or between about 4.5 and about 6.

The provided treatment methods are beneficially very effective in substantially filling targeted endovascular locations to provide an embolization therapy. The fill ratio of the targeted endovascular location following the treatment method can be, for example, greater than about 90%, e.g., greater than about 91%, greater than about 92%, greater than about 93%, greater than about 94%, greater than about 95%, greater than about 96%, greater than about 97%, greater than about 98%, or greater than about 99%. This high filling ratio can advantageously be achieved even when the targeted endovascular location is within, adjacent to, or proximate to a dynamic blood environment having a high mean inlet blood flow velocity. For example, the treatment method can provide an effective embolization therapy even when the mean inlet blood flow velocity within, adjacent to, or proximate to the targeted endovascular site is between about 2 cm/sand about 60 cm/s, e.g., between about 2 cm/sand about 15 cm/s, between about 2.8 cm/sand about 22 cm/s, between about 3.9 cm/sand about 30 cm/s, between about 5.5 cm/sand about 43 cm/s, or between about 7.8 cm/sand about 60 cm/s. In terms of lower limits, the mean inlet blood flow velocity of the dynamic blood environment prior to the introducing of the acid solution can be greater than about 2 cm/s, e.g., greater than about 2.8 cm/s, greater than about 3.9 cm/s, greater than about 5.5 cm/s, greater than about 7.8 cm/s, greater than about 11 cm/s, greater than about 15 cm/s, greater than about 22 cm/s, greater than about 30 cm/s, or greater than about 43 cm/s.

The provided treatment methods also advantageously result in robust and long-lasting embolization therapies. For example, the high fill ratio in the targeted endovascular location following the treatment method can be stably maintained for an extended period under typical physiological conditions. As an indicator of this stability, the blockage formed by an aggregate of the microgels can continue to exhibit self-adhesion in a blood environment at 37° C. for greater than 2 months after completion of the treatment method. Additionally, the shape and the compression modulus of the blockage each independently exhibit less than a 30% change after being in the blood environment at 37° C. for greater than 2 months after completion of the treatment method.

Another advantage of the provided treatment method is that the plurality of microgels, and the blockage formed by an aggregate of the microgels, have excellent biocompatibility within a subject, causing no significant adverse effects. For example, the blockage does not decrease viability of endothelial cells proximate to the target vascular site more than 10% during the first 48 h after the forming of the blockage. Additionally, the blockage does not induce dissolution of more than 10% of red blood cells proximate to the target vascular site during the first 24 h after the forming of the blockage. As another benefit of the biocompatibility, the blockage does not increase procoagulation or anticoagulation of blood proximate to the target vascular site more than 10% during the first 24 h after the forming of the blockage.

### VI. Exemplary Embodiments

The following embodiments are contemplated. All combinations of features and embodiments are contemplated.

Embodiment 1: A pH-responsive self-healing microgel comprising: a polymerization reaction product of two or more polymer precursors and a cross-linker; a magnetic agent; and an imaging agent.

Embodiment 2: An embodiment of embodiment 1, wherein at least one of the two or more polymer precursors comprises an acryloyl group, an acrylamide group, or a combination thereof.

Embodiment 3: An embodiment of embodiment 2, wherein the two or more polymer precursors comprise optionally substituted N-acryloyl 2-glycine, optionally substituted N-acryloyl 4-aminobutyric acid, optionally substituted N-acryloyl 6-aminocaproic acid, optionally substituted N-acryloyl 8-aminocaprylic acid, optionally substituted N-acryloyl 11-aminoundecanoic acid, optionally substituted N-isopropylacrylamide, optionally substituted acrylamide, optionally substituted acrylic acid, or a combination thereof.

Embodiment 4: An embodiment of any one of embodiments 1-3, wherein the polymerization reaction product comprises a plurality of pendant side chains each independently comprising an amide group, a carboxyl group, or a combination thereof.

Embodiment 5: An embodiment of embodiment 4, wherein the plurality of pendant side chains comprises pendant side chains having the structure of the formula:

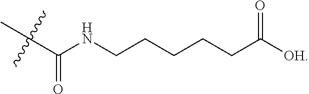 [SMILES: *C(=O)NCCCCCC(=O)O]

Embodiment 6: An embodiment of embodiment 4 or 5, wherein the plurality of pendant side chains forms hydrogen bonds across an interface of the pH-responsive self-adhesive microgel.

Embodiment 7: An embodiment of embodiment 6, wherein the hydrogen bonds form when the pH-responsive self-healing microgel is exposed to a pH of less than or equal to about 6.

Embodiment 8: An embodiment of embodiment 6 or 7, wherein the formed hydrogen bonds are maintained in 0.9 wt % saline at 37° C. for at least about 24 h.

Embodiment 9: An embodiment of any one of embodiments 1-6, wherein the cross-linker comprises an acrylamide group, an acrylate group, a methacrylate group, a vinyl ether group, or a combination thereof.

Embodiment 10: An embodiment of embodiment 9, wherein the cross-linker comprises optionally substituted N,N′-methylenebisacrylamide, optionally substituted poly (ethylene glycol) diacrylate, optionally substituted 1, 4-cyclohexanedimethanol divinyl ether, optionally substituted trimethylolpropane triacrylate, optionally substituted methacrylated gelatin, or a combination thereof.

Embodiment 11: An embodiment of embodiment 10, wherein the cross-linker comprises N,N′-methylenebisacrylamide.

Embodiment 12: An embodiment of any one of embodiments 1-11, wherein the magnetic agent comprises a transition metal.

Embodiment 13: An embodiment of embodiment 12, wherein the magnetic agent comprises a nickel microparticle, an iron microparticle, an iron nanoparticle, an iron oxide microparticle, an iron oxide nanoparticle, a cobalt oxide nanoparticle, an iron-platinum nanoparticle, or a combination thereof.

Embodiment 14: An embodiment of embodiment 13, wherein the magnetic agent comprises a magnetite nanoparticle.

Embodiment 15: An embodiment of any one of embodiments 1-14, wherein the imaging agent comprises a contrast agent.

Embodiment 16: An embodiment of embodiment 15, wherein the imaging agent comprises a barium sulfate nanoparticle, a tantalum microparticle, iodipin, or a combination thereof.

Embodiment 17: An embodiment of embodiment 16, wherein the imaging agent comprises a tantalum microparticle.

Embodiment 18: An embodiment of any one of embodiments 1-17, wherein the pH-responsive self-adhesive microgel comprises between about 10% and about 30% percent of the two or more polymer precursors.

Embodiment 19: An embodiment of any one of embodiments 1-18, wherein the pH-responsive self-healing microgel comprises between about 0.1% and about 1% percent of the cross-linker.

Embodiment 20: An embodiment of any one of embodiments 1-19, wherein the pH-responsive self-healing microgel comprises between about 10% and about 30% percent of the magnetic agent.

Embodiment 21: An embodiment of any one of embodiments 1-20, wherein the pH-responsive self-healing microgel comprises between about 5% and about 15% percent of the imaging agent.

Embodiment 22: An embodiment of any one of embodiments 1-21, wherein the pH-responsive self-healing microgel has an equivalent spherical diameter that is less than about 300 μm.

Embodiment 23: An embodiment of any one of embodiments 1-21, wherein the pH-responsive self-healing microgel has a substantially spherical shape.

Embodiment 24: A population of pH-responsive self-healing microgels comprising the pH-responsive self-adhesive microgel of any one of embodiments 1-23.

Embodiment 25: A system for treating a subject in need of an embolization at a targeted endovascular location, the system comprising: the population of pH-responsive self-healing microgels of embodiment 24; a magnetic control module; and an imaging module.

Embodiment 26: An embodiment of embodiment 25, wherein the magnetic control module is configured to guide the plurality of the pH-responsive self-healing microgels to the targeted endovascular location.

Embodiment 27: An embodiment of embodiment 25 or 26, wherein the magnetic control module comprises a spherical permanent magnet, an electric motor, and a robotic arm.

Embodiment 28: An embodiment of any one of embodiments 25-27, wherein the imaging module is configured to image the plurality of the pH-responsive self-healing microgels within the subject.

Embodiment 29: An embodiment of any one of embodiments 25-28, wherein the imaging module comprises a fluorescence microscopy module, a magnetic resonance imaging module, a positron emission tomography module, an ultrasound imaging module, a fluoroscopy module, a confocal microscopy module, or a combination thereof.

Embodiment 30: An embodiment of embodiment 29, wherein the imaging module comprises an ultrasound imaging module and a fluoroscopy module.

Embodiment 31: An embodiment of any one of embodiments 25-30, wherein the system further comprises a catheter.

Embodiment 32: An embodiment of embodiment 31, wherein the catheter is configured to administer the plurality of the pH-responsive self-healing microgels to the subject.

Embodiment 33: A method of treating a subject in need of an embolization at a targeted endovascular location in the subject, the method comprising: administering the population of pH-responsive self-healing microgels of claim 24 to the subject; applying a magnetic field to at least a portion of the subject; guiding the population of pH-responsive self-healing microgels to the targeted endovascular location using the magnetic field; and introducing an amount of an acidic solution to the targeted endovascular location, wherein the amount is effective in inducing formation of hydrogen bonds between pH-responsive self-healing microgels of the population of pH-responsive self-healing microgels.

Embodiment 34: An embodiment of embodiment 33, wherein the method further comprises determining positional information for the population of pH-responsive self-healing microgels by imaging the population of pH-responsive self-healing microgels in the subject.

Embodiment 35: An embodiment of embodiment 34, wherein the guiding is based on the positional information.

Embodiment 36: An embodiment of embodiment 34 or 35, wherein the imaging comprises real-time imaging.

Embodiment 37: An embodiment of any one of embodiments 34-36, wherein the imaging comprises fluorescence microscopy imaging, magnetic resonance imaging, positron emission tomography imaging, ultrasound imaging, a fluoroscopy imaging, confocal microscopy imaging, or a combination thereof.

Embodiment 38: An embodiment of embodiment 37, wherein the imaging comprises ultrasound imaging and fluoroscopy imaging.

Embodiment 39: An embodiment of any one of embodiments 33-38, wherein the applying of the magnetic field is performed using a magnetic control module comprising a spherical permanent magnet, an electric motor, and a robotic arm.

Embodiment 40: An embodiment of any one of embodiments 33-39, wherein the administering of the population of pH-responsive self-healing microgels is performed using a catheter.

Embodiment 41: An embodiment of any one of embodiments 33-40, wherein the acidic solution has a pH less than about 6.

Embodiment 42: An embodiment of any one of embodiments 33-41, wherein the targeted endovascular location is within, adjacent to, or proximate to an aneurysm of the subject.

Embodiment 43: An embodiment of any one of embodiments 33-42, wherein, prior to the introducing of the acidic solution, the targeted endovascular location is within, adjacent to, or proximate to a dynamic blood environment having a mean inlet blood flow velocity between about 2 cm/sand about 60 cm/s.

Embodiment 44: An embodiment of any one of embodiments 33-43, wherein, subsequent to the introducing of the acidic solution, the targeted endovascular location has a filling ratio greater than about 90%

### EXAMPLES

The present disclosure will be better understood in view of the following non-limiting examples. The following examples are intended for illustrative purposes only and do not limit in any way the scope of the present disclosure.

### Example 1. PH-Responsive Self-Healing Properties of Provided Microgels

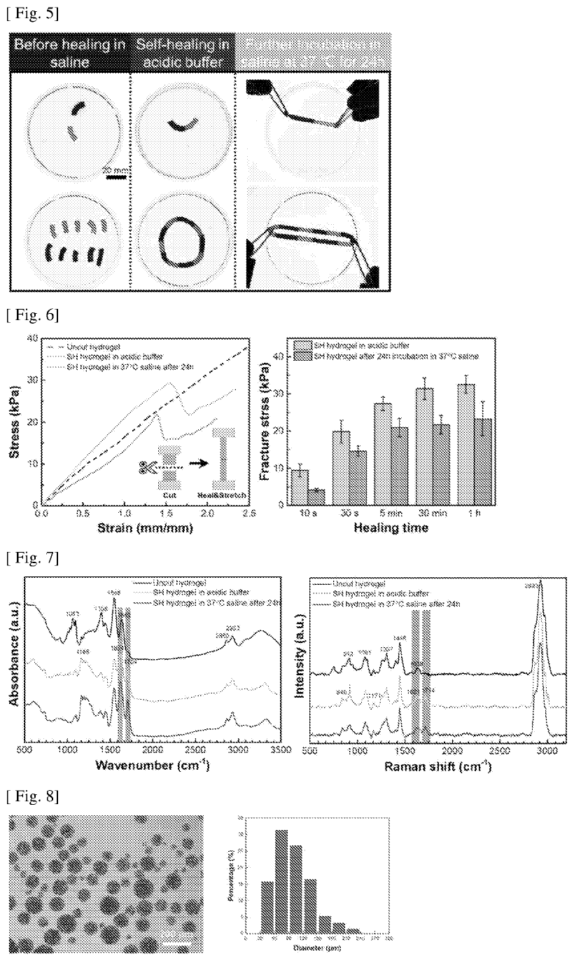

In the experiment depicted in FIG. 5, several hydrogel segments dyed with red and blue colors for differentiation were prepared and conserved in saline. Once immersed in acidic aqueous environment (ascorbic acid buffer solution with a pH value of 5), two hydrogels brought in contact welded rapidly to each other within 10 s and exhibited a strong interface capable of withstanding manual stretching. By repeating this process, multiple hydrogels could be readily assembled into a ring-like structure, demonstrating the excellent pH-responsive self-healing behavior of the provided hydrogel. Moreover, after removing the acidic buffer and soaking the hydrogel ring in saline at 37° C., the adhesion between adjacent segments was found to still take effect for at least 24 h, indicating the stable healing performance of prepared hydrogel material in a simulated biological fluid (SBF).

### Example 2. Mechanical Properties of Provided Microgels

The healing performance of the provided hydrogel was further quantitatively evaluated by mechanical stretch testing. Hydrogel cylinders were first fabricated using a tubular mold, and then fixed to a universal tensile testing machine. Results demonstrated a pristine fracture stress of 57.1 kPa and an elastic modulus of 15.9 kPa. Subsequently, the hydrogels were cut off in the middle position, and healed in acidic buffer for 5 min, followed by incubation in saline at 37° C. for 24 h. As shown in FIG. 6, the maximum stress required to break the hydrogel just after this healing process was approximately 29.2 kPa, indicating the efficient recovery of more than 50% of the tensile strength in a short time. Further incubating healed hydrogel in SBF only induced a slight deterioration of the fracture stress to approximately 21.7 kPa instead of the complete failure of healing behavior. These results demonstrate the suitability of the provided materials for practical biomedical applications in biological environments.

The temporal dependence of the hydrogel healing process was also investigated. The results plotted in FIG. 6 show that adhesion performance elevated continuously with an increase of healing time, and then gradually stabilized after healing for 30 min. Especially for those hydrogels incubated in SBF for 24 h, the average fracture stress only changed from 20.9 to 23.3 kPa when extending the healing time from 5 min to 1 h. Notably, the hydrogels exposed to acidic buffer for only 30 s possessed an interfacial adhesion strength as high as 14.6 kPa, demonstrating the fast healing ability of the provided hydrogel material.

### Example 3. Hydrogen Bond Characterization in Provided Microgels

To confirm the self-healing behavior was regulated and controlled by the formation of hydrogen bonding, Fourier transform infrared spectroscopy-attenuated total reflectance (FTIR-ATR) and Raman spectroscopy were conducted. As shown in FIG. 7, when the hydrogel was treated with acidic buffer, new prominent peaks emerged at 1704 cm⁻¹ (FTIR-ATR) and 1714 cm⁻¹ (Raman), indicating the presence of hydrogen-bonded terminal carboxylic acid group. Additionally, the FTIR-ATR band at 1640 cm⁻¹ and Raman band at 1638 cm⁻¹ assigned to amide I group downshifted to 1624 and 1621 cm⁻¹, respectively, demonstrating the enhanced H bonding to the amide group. Such spectroscopic analyses suggested the formation of two hydrogen bonding types in the polymeric matrix under acidic condition, i.e., the face-on interaction between opposite carboxyl groups and the interleaved interaction between carboxyl and amide groups (FIG. 4). Without being bound by a particular theory, the results suggest that these hydrogen bonding types constitute the basic self-healing mechanism of the provided hydrogel. After the hydrogel was completely rinsed and incubated in saline at 37° C. for 24 h, no significant difference could be observed in the FTIR-ATR and Raman spectra. These findings are consistent with the stable welding performance in SBF demonstrated above, show the advantages of the provided hydrogel for the development of embolic microrobots for on-demand embolization treatment of aneurysm.

### Example 4. Physical Stability of Provided Microgels

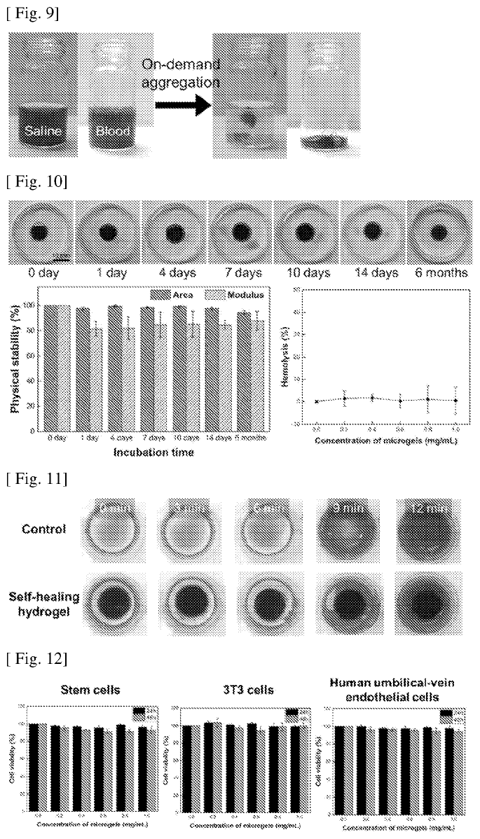

FIG. 8 shows the morphologies of prepared microgels, confirming successful batch preparation of microrobots with a regular and spherical shape. The quantitative statistics demonstrate that the provided microgels can have an average diameter of approximately 110 μm. This average size can be easily modulated by adjusting the stirring rate or the viscosity of aqueous phase during the emulsion process, facilitating the specific and customizable embolization of vascular sac with different sizes. Further, due to the presence of the self-healing hydrogel matrix, the microgels exhibit an advantageous self-adhesive property in a pH-responsive manner. As shown in FIG. 9, a crowd of microgels can adhere with each other rapidly (e.g., within seconds) to weld into an agglomerate once acidic buffer is injected into the suspension. This observed behavior also took effect in a blood environment.

For use in long-term embolization treatments, the embolic microrobots should beneficially possess physical stability and compatibility with the surrounding biological environment within a subject. The provided embolic material composed of self-healing hydrogel matrix, magnetic agent, and imaging agent was molded into small discs (10 mm in diameter) for convenience in investigating these stability and biocompatibility properties. FIG. 10 shows that hydrogel discs immersed in anti-coagulated porcine blood for different times exhibited no significant shape change, even after 6 months. FIG. 10 also presents results from quantitative analyses of the area and compression modulus of the immersed discs. The area was found to only decrease by approximately 5.5% after long-term storage in blood. The corresponding modulus was reduced to approximately 82.5% of the initial value at first day due to the exchange of blood into hydrogel matrix, and then remained mostly stable for the following 180 days. These results demonstrate that the provided material for embolic microrobots can maintain a substantially constant shape, size, and mechanical properties for an extended term, and thus exhibit excellent physical stability.

### Example 5. Biocompatibility and Detectability of Provided Microgels

In further tests, blood cells were centrifuged from porcine blood and incubated with microgels in phosphate buffer solution. After 24 h, the supernatant was collected and characterized to verify whether the dissolution of blood cells occurred. Results showed a hemolysis rate that was always lower than 5% regardless of microgel concentration, indicating the provided microrobots do not impose harmful effect on blood cells.

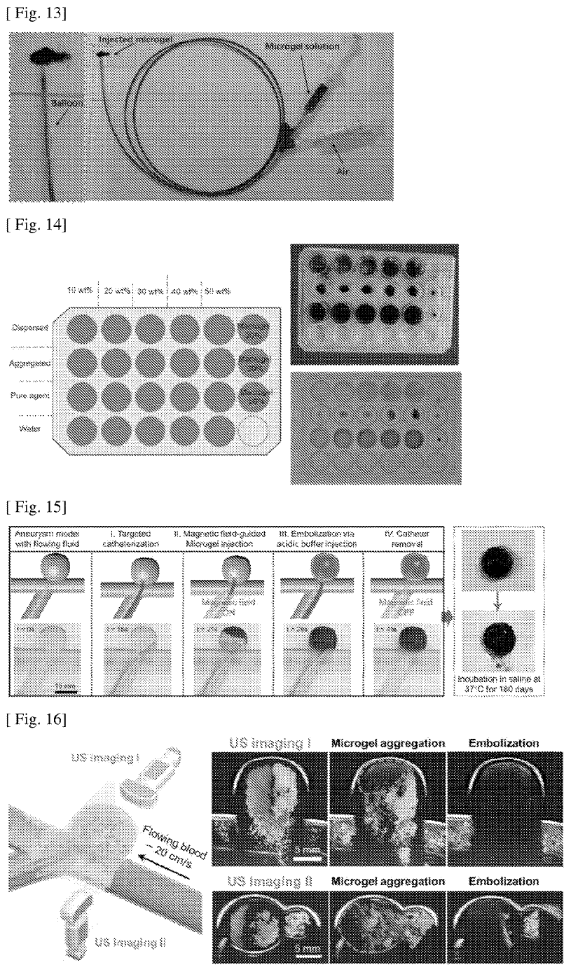

Because embolic microrobots for embolization treatment operate in blood vessels and keep constant contact with blood, the provided materials should be bio- and hemo-compatible. FIG. 11 shows the influence of the provided microrobots on blood coagulation. A disc-shaped embolic material was immersed into porcine blood, followed by the addition of calcium ions to induce blood coagulation. Compared to the blank group, the status of blood contacting with embolic hydrogel presented a similar variation tendency, and no significant difference was observed in the blood clotting time. These results confirm that the provided microrobots do not cause either blood procoagulation or anticoagulation. Microgels were also prepared and co-cultured with different types of normal cells for 48 h. As shown in FIG. 12, compared to the control group, the addition of microgels did not induce any change in the viability of stem cells, 3T3 cells, and human umbilical vein endothelial cells (HUVECs), even with a concentration as high as 1.0 mg/mL. These results demonstrate the negligible cytotoxicity of the provided microrobots. In summary, the provided microrobots can be smoothly deployed via a clinical catheter (FIG. 13), demonstrate long-term physical stability, and exhibit excellent bio- and hemo-compatibility with physiological blood environments.

Also beneficially, following incorporation of Ta microparticles into the provided microgel, fluoroscopy imaging contrast proportional to the Ta concentration was detected. FIG. 14 shows fluoroscopy images indicating the feasibility of this approach for imaging and tracking the provided microrobots inside a living body, e.g., inside a subject being treated with the microrobots. For this additional reason, the disclosed materials and fabrication methods provide spherical embolic microrobots capable of pH-responsive self-adhesion, and in vivo imaging. These advantageous characteristics further demonstrate the suitability of the disclosed materials and methods to provide embolic agents.

### Example 6. Blockage Formation in Aneurysm with Provided Microgels

FIG. 15 shows a demonstration of the provided on-demand embolization process in an aneurysm replica and a dynamic fluidic environment. The inlet mean flow velocity was set to be 20 cm/s, and a clinical catheter was first inserted through a branched pipe with its tip placing at the aneurysm neck (stage I). Then, a dynamic external magnetic field was applied via the robotic magnet, followed by the deployment of swarming microgels, resulting in the magnetically guided aggregation of embolic microrobots in the aneurysm sac (stage II). After accumulating sufficient microgels, an acidic buffer solution was released via catheter to trigger the pH-responsive self-adhesive behavior of microgels, which caused the welding of swarming microrobots into an aggregation to occlude the sac (stage III). Finally, the catheter and robotic magnet were removed (stage IV), indicating the accomplishment of on-demand aneurysm embolization. After embolization, the aneurysm replica was dissected, followed by removal and storage of the blockage welded by embolic microrobots in saline at 37° C. No significant difference in the morphology could be observed even after 6 months, confirming the long-term stability of embolization effect.

The applicability of the provided microrobotic embolization strategy for aneurysm treatment was further verified in a blood environment. Due to the opaqueness of blood, ultrasound (US) imaging was first employed to monitor the embolization process instead of optical imaging. FIG. 16 presents associated ultrasound images from this study. Except for standard B mode, US Doppler imaging, a noninvasive medical imaging strategy compatible with magnetic field, was applied to estimate the flowing state of blood. In this manner, US imaging not only allowed for monitoring of the instant operation progress, but also enabled the visualization of blood occluding conditions in the aneurysm sac, thus serving as a useful tool for evaluating embolization performance. Two US imaging planes (I and II) were adopted to reflect the real-time situations on the horizontal and vertical cross sections of aneurysm sac, respectively. Before the deployment of swarming microgels, obvious Doppler red and blue signals corresponding to the red blood cells moving toward and away from the US source, respectively, could be found distributed in the whole aneurysm sac and branched pipe, indicating robust blood circulation in the aneurysm replica. When swarming microgels were released via catheter and magnetically aggregated, a B mode shadow signal appeared (i.e., the area surrounded by the dashed line) and continuously pushed the boundary of Doppler signals, demonstrating attenuation of the blood flow in aneurysm sac. After an acidic buffer was injected to achieve the welding of swarming microgels, the Doppler signals inside the cavity completely vanished both for US imaging planes I and II, indicating there was no longer blood flowing into the aneurysm.

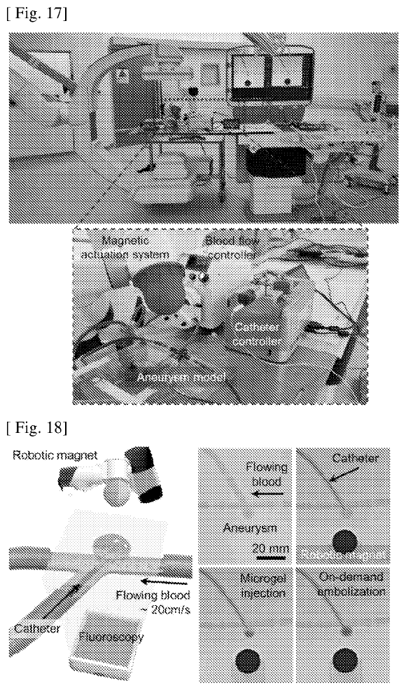

To further expand the provided microrobotic embolization strategy to in vivo scenarios, c-arm fluoroscopy, a general medical imaging method for interventional therapy, was used to test the embolization process. FIG. 17 depicts the experimental setup used for this study. By controlling the rotating magnet integrated on a robotic arm, the deployed swarming microgels were concentrated in the aneurysm sac without unintentional leakage, even in the dynamic blood environment with a mean flowing velocity of 20 cm/s. As shown in FIG. 18, the controlled concentration of the deployed microgels could be clearly monitored via fluoroscopy. These results demonstrated that the provided swarming self-adhesive microgels function in physiological blood environment for real-time imaging-guided aneurysm embolization.

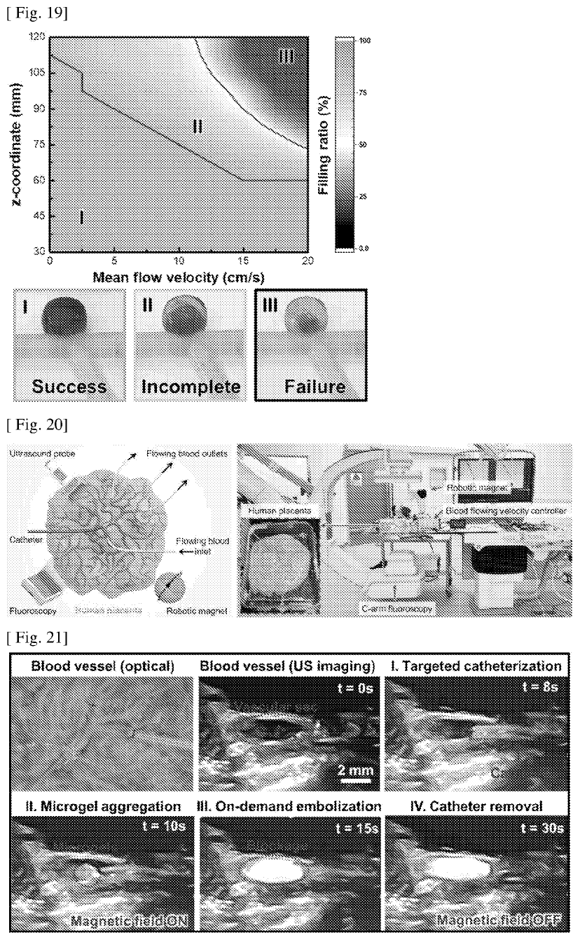

FIG. 19 shows results from a further experimental validation of the magnetic aggregation performance of swarming microrobots in an aneurysm model under flowing condition. As shown in region I of FIG. 19, when the z-coordinate was small enough (i.e., the robotic magnet was close enough to aneurysm), the aneurysm sac could be completely filled, even when an inlet mean flowing velocity as high as ~20 cm/s was applied. With the increase of z-coordinate, the maximum flowing velocity that the swarming microgels could tolerate gradually decreased. When the actual flowing velocity exceeded the critical value, partial microgels may no longer remain stable in the sac, leading to the incomplete filling of aneurysm as shown in region II. Further shifting the robotic magnet away from aneurysm induces a notable deterioration in the aggregation performance. Cases in which the filling ratio was lower than 50% (region III) were classified as failure. The observed results were scalable for the magnetic control system with varied field strength and gradient, with a larger and stronger magnet generally facilitating better magnetic aggregation effects.

### Example 7. Blockage Formation in Placenta with Provided Microgels

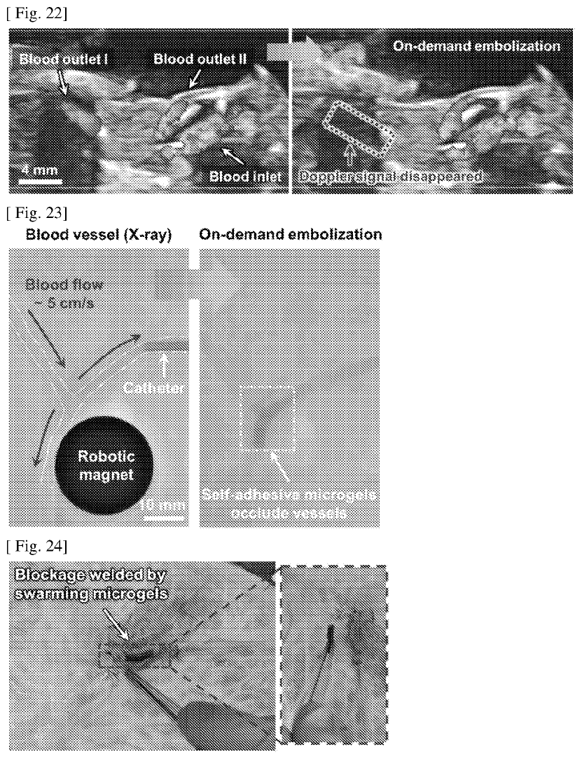

The effectiveness of the provided microrobotic strategy in blood vessel was further verified by conducting the embolization process in a human placenta ex vivo under the guidance of ultrasound and fluoroscopy imaging. FIG. 20 presents the schematic and experimental setup used for this study. The placenta filled with flowing blood was selected to emulate a vascular system due to the presence of abundant blood vessels as shown in the inset of FIG. 20. A clinical catheter mounted on a syringe pump was inserted into the blood vessel for microgel deployment, a robotic arm-integrated rotating magnet was established to control the magnetic field, and a peristaltic pump connected to four arterial blood vessels was responsible for the construction of dynamic blood circulation within the placenta. The mean blood flow velocity of the inlet vessel was set to be 5 cm/s, as a larger flowing velocity with higher pressure might induce rupture of the blood vessels in the placenta. FIG. 21 shows the real-time US B mode images of the entire embolization process in a specific blood artery, including catheterization, microgel aggregation under magnetic field, on-demand embolization via acidic buffer injection, catheter removal and magnetic field turnoff. FIG. 22 presents US Doppler images during the operation, showing that the Doppler signals of flowing blood in the distal vessel (blood outlet I) disappeared after the embolization process, indicating efficient blockage of the targeted artery. The images of FIG. 23 confirm that such procedures can be clearly monitored by c-arm fluoroscopy inside biological tissue using the intrinsic X-ray imaging contrast of the provided microgels. As shown in FIG. 24, dissection of the occluded blood artery performed after embolization revealed a blockage welded by swarming self-adhesive microgels in the vessel. Firm adhesion between the provided microgels was achieved, allowing the blockage to be tweezed without fragmentation. Together, these results demonstrate the practicability of the provided microrobotic embolization platform for on-demand occlusion of real blood vessels in ex vivo human organs, as well as the applicability of the provided ultrasound and fluoroscopy imaging strategies for monitoring and evaluating the performance of provided operation procedures.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only and is not intended to limit the disclosure. In addition, each reference provided herein is incorporated by reference in its entirety for all purposes to the same extent as if each reference was individually incorporated by reference.

1. A pH-responsive self-healing microgel comprising:
a polymerization reaction product of two or more polymer precursors and a cross-linker;
a magnetic agent; and
an imaging agent.

2. The pH-responsive self-healing microgel of claim 1, wherein the polymerization reaction product comprises a plurality of pendant side chains having the formula:

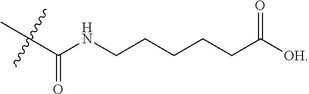 [SMILES: *C(=O)NCCCCCC(=O)O]

3. The pH-responsive self-healing microgel of claim 1, wherein the cross-linker comprises N,N′-methylenebisacrylamide.

4. The pH-responsive self-healing microgel of claim 1, wherein the magnetic agent comprises a magnetite nanoparticle.

5. The pH-responsive self-healing microgel of claim 1, wherein the imaging agent comprises a tantalum microparticle.

6. The pH-responsive self-healing microgel of claim 1, wherein the pH-responsive self-adhesive microgel comprises between about 10% and about 30% percent of the two or more polymer precursors.

7. The pH-responsive self-healing microgel of claim 1, wherein the pH-responsive self-healing microgel comprises between about 0.1% and about 1% percent of the cross-linker.

8. The pH-responsive self-healing microgel of claim 1, wherein the pH-responsive self-healing microgel comprises between about 10% and about 30% percent of the magnetic agent.

9. The pH-responsive self-healing microgel of claim 1, wherein the pH-responsive self-healing microgel comprises between about 5% and about 15% percent of the imaging agent.

10. The pH-responsive self-healing microgel of claim 1, wherein the pH-responsive self-healing microgel has an equivalent spherical diameter that is less than about 300 μm.

11. A population of pH-responsive self-healing microgels comprising the pH-responsive self-adhesive microgel of claim 1.

12. A system for treating a subject in need of an embolization at a targeted endovascular location, the system comprising:
the population of pH-responsive self-healing microgels of claim 11;
a magnetic control module; and
an imaging module.

13. The system of claim 12, wherein the magnetic control module is configured to guide the plurality of the pH-responsive self-healing microgels to the targeted endovascular location.

14. The system of claim 12, wherein the magnetic control module comprises a spherical permanent magnet, an electric motor, and a robotic arm.

15. The system of claim 12, wherein the imaging module is configured to image the plurality of the pH-responsive self-healing microgels within the subject.

16. The system of claim 12, wherein the imaging module comprises an ultrasound imaging module and a fluoroscopy module.

17. A method of treating a subject in need of an embolization at a targeted endovascular location in the subject, the method comprising:
administering the population of pH-responsive self-healing microgels of claim 11 to the subject;
applying a magnetic field to at least a portion of the subject;
guiding the population of pH-responsive self-healing microgels to the targeted endovascular location using the magnetic field; and
introducing an amount of an acidic solution to the targeted endovascular location, wherein the amount is effective in inducing formation of hydrogen bonds between pH-responsive self-healing microgels of the population of pH-responsive self-healing microgels.

18. The method of claim 17, wherein the method further comprises determining positional information for the population of pH-responsive self-healing microgels by imaging the population of pH-responsive self-healing microgels in the subject.

19. The method of claim 18, wherein the guiding is based on the positional information.

20. The method of claim 18, wherein the imaging comprises real-time imaging.
